(19) United States
(12) Patent Application Publication
(10) Pub. No.: US 2020/0405619 A1
(43) Pub. Date: Dec. 31, 2020

(54) ORAL HYGIENE COMPOSITIONS

(71) Applicant: Javad Ghoreishi, Brookline, MA (US)

(72) Inventor: Javad Ghoreishi, Brookline, MA (US)

(21) Appl. No.: 16/678,054

(22) Filed: Nov. 8, 2019

Related U.S. Application Data
(60) Provisional application No. 62/868,978, filed on Jun. 30, 2019.

(51) Int. Cl. A61K 8/92; A61K 8/21; A61K 8/60; A61K 8/37; A61K 8/25; A61K 8/73; A61K 8/362; A61K 8/28

(52) U.S. Cl. CPC A61K 8/922; A61K 8/21; A61K 8/60; A61K 8/28; A61K 8/25; A61K 8/73; A61K 8/362; A61K 8/37

(57) ABSTRACT

An oral hygiene composition which relates to oral hygiene compositions. More particularly, the present disclosure relates to a toothpaste which combines stannous fluoride and tea tree oil (Melaleuca). The composition prevents tooth decay and new cavity formation on enamel and dentin, reducing dentinal hypersensitivity when used regularly. The present disclosure helps prevent gum disease (Gingivitis) bad breath and improves oral hygiene and overall health.

### CROSS REFERENCE TO RELATED APPLICATION

The present application is related to and claims priority to U.S. Provisional Patent Application No. 62/868,978 filed 2019 Jun. 30, which is incorporated by reference herein in its entirety.

### BACKGROUND OF THE DISCLOSURE

The following includes information that may be useful in understanding the present disclosure. It is not an admission that any of the information provided herein is prior art nor material to the presently described or claimed disclosures, nor that any publication or document that is specifically or implicitly referenced is prior art.

### 1. FIELD OF THE DISCLOSURE

The present disclosure relates generally to the field of oral hygiene compositions and more specifically relates to toothpaste compositions and packaging.

### 2. DESCRIPTION OF RELATED ART

Many people suffer from tooth decay, which can lead to root canals and gum disease. Not only are these conditions painful and can keep people up at night, they can also lead to infections and a decline in overall health. Some people may use one of the many toothpaste products on the market in an attempt to overcome these issues, but these products fail to combine fluoridation with anti-viral, anti-bacterial, anti-fungal, and anti-septic to clean the mouth. A suitable solution is desired.

### BRIEF SUMMARY OF THE DISCLOSURE

The present disclosure provides oral hygiene compositions. The general purpose of the present disclosure, which will be described subsequently in greater detail, is to provide an oral hygiene composition. More particularly, the present disclosure relates to toothpaste compositions that combine stannous fluoride and tea tree oil (melaleuca). The composition prevents tooth decay and new cavity formation on enamel and dentin, reducing dentinal hypersensitivity when used regularly. The present disclosure helps prevent gum disease (gingivitis) bad breath and improves oral hygiene and overall health. The toothpaste composition of this disclosure saves people a considerable amount of time and frustration at the dentist, while leaving a healthy mouth with a refreshed clean feeling.

An oral hygiene composition is disclosed herein. In accordance with a preferred embodiment hereof, this disclosure provides an oral hygiene composition for promoting oral health comprising: an orally compatible carrier; and an admixture consisting essentially of a therapeutically effective amount of SnF₂ (stannous fluoride); and a therapeutically effective amount of Melaleuca (tea tree oil). Moreover, it provides such an oral hygiene composition, wherein the therapeutically effective amount of SnF₂ (stannous fluoride) consists of about 0.454 percent by volume. Additionally, it provides such an oral hygiene composition, wherein the therapeutically effective amount of Melaleuca (tea tree oil) consists of between about 1 percent and about 5 percent by volume. Also, it provides such an oral hygiene composition, wherein the orally compatible carrier consists of a semi-liquid aqueous paste. In addition, it provides such an oral hygiene composition, wherein the orally compatible carrier consists of at least one sugar substitute selected from the group consisting essentially of sorbitol, saccharin, and sucralose. Further, it provides such an oral hygiene composition, wherein the orally compatible carrier comprises components selected from the group consisting essentially of flavorants, hydrated silica, sodium lauryl sulfate, carrageenan, sodium gluconate, xanthan gum, cocamidopropyl betain, zinc citrate, sodium hydroxide, and titanium dioxide. Even further, the disclosure may include packaging configured to package the oral hygiene composition; wherein the packaging is selected from a squeezable plastic tube, a squeezable aluminum tube, and a squeezable laminated tube constructed from aluminum and plastic.

A method of producing oral hygiene composition is also disclosed herein. The method of producing the oral hygiene composition may comprise the steps of: providing an aqueous orally compatible carrier; forming an admixture may comprise a therapeutically effective amount of SnF₂ (stannous fluoride) and a therapeutically effective amount of tea tree oil; and combining the admixture and the aqueous orally compatible carrier to form such oral hygiene composition. Even further, such a method of producing the oral hygiene composition further may include the step of formulating the aqueous orally compatible carrier from at least one member selected from the group consisting essentially of: water; at least one sugar substitute; hydrated silica; sodium lauryl sulfate; carrageenan; sodium gluconate; at least one flavorant; xanthan gum; cocamidopropyl betain; zinc citrate; and titanium dioxide.

For purposes of summarizing the disclosure, certain aspects, advantages, and novel features of the disclosure have been described herein. It is to be understood that not necessarily all such advantages may be achieved in accordance with any one particular embodiment of the disclosure. Thus, the disclosure may be embodied or carried out in a manner that achieves or optimizes one advantage or group of advantages as taught herein without necessarily achieving other advantages as may be taught or suggested herein. The features of the disclosure which are believed to be novel are particularly pointed out and distinctly claimed in the concluding portion of the specification. These and other features, aspects, and advantages of the present disclosure will become better understood with reference to the following drawings and detailed description.

### BRIEF DESCRIPTION OF THE DRAWINGS

The figures which accompany the written portion of this specification illustrate embodiments and methods of producing the present disclosure, oral hygiene compositions, constructed and operative according to the teachings of the present disclosure.

FIG. 1 is a diagram illustrating the primary constituent components of the oral hygiene composition, according to an embodiment of the disclosure.

FIG. 2 shows a front view of product packaging configured to package the oral hygiene composition of FIG. 1, according to an embodiment of the present disclosure.

FIG. 3 shows a series of sectional views, enlarged for clarity of description, illustrating the product packaging of FIG. 2 in greater detail, according to embodiments of the present disclosure.

FIG. 4 is a flow diagram illustrating a method of producing the oral hygiene composition of FIG. 1, according to an embodiment of the present disclosure.

The various embodiments of the present disclosure will hereinafter be described in conjunction with the appended drawings, wherein like designations denote like elements.

### DETAILED DESCRIPTION

As discussed above, embodiments of the present disclosure relate to an oral hygiene composition and more particularly to oral hygiene compositions as used to improve oral health.

Generally, this innovative oral hygiene composition features a toothpaste (any color), made from various ingredients including stannous fluoride and tea tree oil (Melaleuca). The oral hygiene compositions of the present disclosure are intended primarily for adults and for children age three and up after consulting with the child's pediatrician. The toothpaste formulations of the present disclosure are customarily used with a toothbrush. The present disclosure relates to oral hygiene compositions. More particularly, the present disclosure prevents tooth decay and helps prevent gum disease (Gingivitis) for improved oral hygiene and overall health. The toothpaste saves people a considerable amount of time and frustration at the dentist, while leaving a healthy mouth with refreshed clean feeling. The composition of this toothpaste prevents formation of cavities on teeth, enamel, and dentin, and reduces dentinal hypersensitivity when used regularly. The disclosed composition improves overall health by eliminating bacteria, virus, fungus and acting as a disinfectant. The toothpaste may be offered in a squeezable tube for maximum convenience.

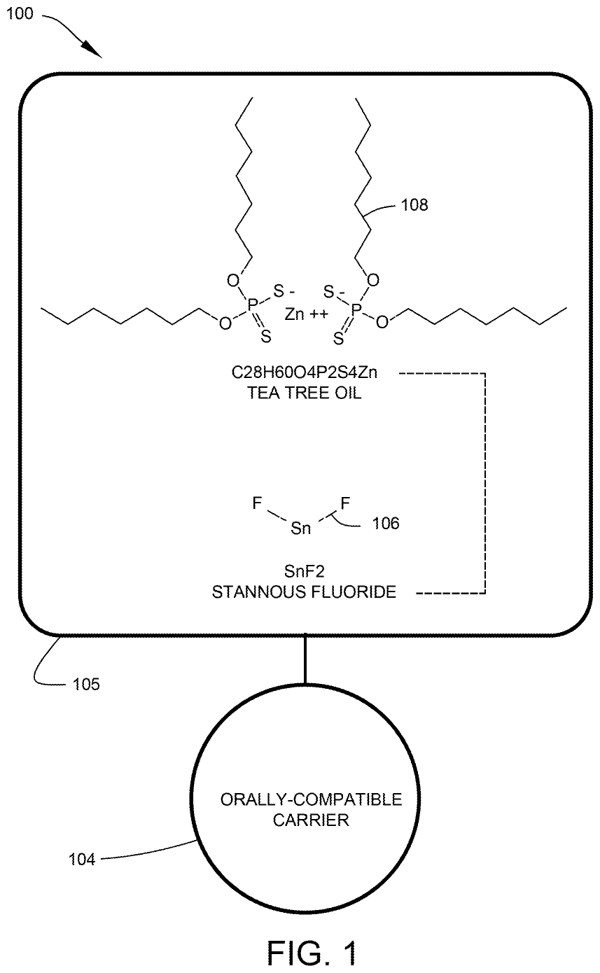

Referring now more specifically to the drawings by numerals of reference, there is shown in FIGS. 1-3, various arrangements of an oral hygiene composition 100 and associated product packaging 102. FIG. 1 illustrates the main constituents of the oral hygiene composition 100, according to embodiments of the present disclosure. As illustrated, the oral hygiene composition 100 may include an orally compatible carrier 104 combined with an admixture 105 consisting essentially of a therapeutically effective amount of SnF₂ (stannous fluoride 106); and a therapeutically effective amount of Melaleuca (tea tree oil 108).

Stannous fluoride 106, or Tn(II) fluoride, is an FDA-approved compound used in toothpastes for the prevention of gingivitis, dental infections, cavities, and to relieve dental hypersensitivity. The anti-plaque, anti-gingivitis, anti-sensitivity, and anti-cavity efficacy of stannous fluoride has been demonstrated in research.

Tea tree oil 108, also known as melaleuca oil, is a volatile essential oil derived mainly from the Australian native tree Melaleuca altemifolia. Tea tree oil 108 is customarily produced by steam distillation of the leaves and terminal branches of M. altemifolia. Once condensed, the clear to pale yellow oil is separated from the aqueous distillate.

Clinical investigation has identified in tea tree oil 108, broad-spectrum antibacterial, antifungal, antiviral, and antiprotozoal properties. For example, the mechanism of action of against bacteria is believed to be related to the ability of the compound to impact bio-membrane integrity and function. The mechanism of antifungal action appears to be associated with the inhibiting of germ tube formation and mycelial conversion. The mechanism of antiviral action is the subject of continued researched; however, initial studies suggest that tea tree oil may act against a broad range of viruses, including enveloped and non-enveloped types. The antiprotozoal action tea tree oil has been demonstrated clinically; however, the mechanism of action also remains under study.

Tea tree oil 108 is generally composed of terpene hydrocarbons, mainly monoterpenes, sesquiterpenes, and associated alcohols. The standard molecular formula of Tea tree oil 108 is C₂₈H₆₀O₄P₂S₄Zn. Tea tree oil 108 suitable for use in the present disclosure may generally conform to the international standard ISO 4730 entitled “Oil of Melaleuca, Terpinen-4-ol type”. ISO 4730 generally specifies that only the terpinen-4-ol chemotype is to be used to ensure the terpinen-4-ol level in the Tea tree oil 108 of the present disclosure exceeds a minimum of about 35.00%. ISO 4730 further specifies the levels of 15 of approximately 100+ components of pure Tea tree oil 108, as indicated in TABLE 1.

TABLE 1
Typical ISO 4730 composition of M. alternifolia tea tree oil

ISO 4730 range

| Component | Minimum % | Maximum % |
|---|---|---|
| α-Pinene | 1 | 4 |
| Sabinene | Trace <0.01% | 3.5 |
| α-Terpineol | 6 | 12 |
| Limonene | 0.5 | 1.5 |
| ρ-Cymene | 0.5 | 8 |
| 1,8-Cineole | Trace <0.01% | 10.0 |
| γ-Terpinene | 14 | 28 |
| Terpinolene | 1.5 | 5 |
| Terpinen-4-ol | 35 | 48 |
| α-Terpineol | 2 | 5 |
| Aromadendrene | 0.2 | 3 |
| Ledene | 0.1 | 3 |
| δ-Cadinene | 0.2 | 3 |
| Globulol | Trace <0.01% | 1 |
| Viridiflorol | Trace <0.01% | 1 |

As indicated in TABLE 1, ISO 4730 provides a range (maximum and minimum) for the objective parameters to allow for the variation that occurs in natural products. This natural variability may be the result of slight differences in growing conditions from year to year, minor genetic variation between plants and differing distillation techniques used to extract the oil.

Admixture 105 was formulated following extensive experimentation and testing. The research evaluation criteria included factors relating to overall oral health in human subjects, with a specific focus on evaluating the management and prevention of dental caries and gingivitis.

Efficacy data collected during observational testing was evaluated by the principal investigator in accordance with defined protocols. The results of the investigation suggest that synergistic combinations of stannous fluoride 106 and tea tree oil 108 provide improvements in overall oral health and the management and prevention of dental caries and gingivitis. Combinations of alternate fluoride-ion sources with other active compounds were evaluated during the study. Interactions of stannous fluoride with other compounds were also assessed. In each case, the principal investigator noted superior outcomes in individuals administered compositions containing admixtures combining stannous fluoride 106 and tea tree oil 108. The research suggests a measurable relationship exists between the presence of admixture 105 and the enhanced efficacy of oral hygiene compositions 100 containing admixture 105. It should be noted that this result was unexpected at the beginning of the study, principally due to the inherent complexity of interactions between the constituent molecules in vivo.

Although the specific therapeutic mechanism of the admixture 105 remains uncertain, it is surmised that the synergistic combination of stannous fluoride 106 and tea tree oil 108 may function to enhance the broad-spectrum anti-viral, anti-bacterial, anti-fungal, and anti-septic effect of both constituent compounds. Moreover, the synergistic properties of the disclosed admixture 105 may allow for increased enamel mineralization by the stannous fluoride component by beneficially altering the poly-microbial biofilm in the mouth.

The orally compatible carrier 104 may comprise a semi-liquid aqueous paste or gel. Such pastes or gels may include abrasive agents, detergents, surfactants, flavoring agents, humectants, thickening agents or binders, stabilizers, foaming agents, preservatives, PH modifiers, peroxides, and the like. Upon reading this specification, it should be appreciated that, under appropriate circumstances, considering such issues as user preferences, design preference, chemical requirements, marketing preferences, cost, available materials, technological advances, etc., other composition ingredients such as, for example, colorants, medications, humectants, such as glycerol, propylene glycol and sorbitol, propylene glycol, hydrogen and carbamide peroxides to help reduce intrinsic stains, etc., may be sufficient.

Flavoring agents, and non-caloric sweeteners may be added to the orally compatible carrier 104 to improve taste. It is noted that sugar and other cariogenic ingredients are not permitted in any ADA-accepted toothpaste; thus, the orally compatible carrier 104 may include at least one sugar substitute. Such sugar substitutes may be selected from the group consisting essentially of sorbitol, sodium saccharin (benzoic sulfimide), and sucralose.

Examples of suitable flavoring agents include oil of spearmint, peppermint, wintergreen, clove, sassafras, sage, eucalyptus, cinnamon, marjoram, lemon, orange, methyl salicylate, and the like.

Abrasive agents may include hydrated silica used as a fine gel abrasive, frequently combined with softer calcium carbonate (from chalk) the abrasives help to remove plaque and may help whiten teeth by physically removing surface stains. Other abrasive agents may include other modified silica abrasives, enzymes, calcium carbonate, dehydrated silica gels, hydrated aluminum oxides, magnesium carbonate, phosphate salts and various other silicates.

The orally compatible carrier 104 may further include one or more detergents to create foaming action that may help increase the solubility of plaque and accretions during brushing. Detergents suitable for use in present disclosure include sodium lauryl sulfate, which may act as an anionic detergent, surfactant, and an effective foaming agent. Other detergents may include sodium N-lauryl sarcosinate.

The orally compatible carrier 104 may further include one or more thickening agents or binders to stabilize the composition. Thickening agents suitable for use in the present disclosure may include carrageenan and xanthan gum. Xanthan gum is a multifunctional ingredient functioning as a thickener, a stabilizer, emulsifier, and suspending agent. Other thickening agents may include mineral colloids, natural gums, seaweed colloids or synthetic cellulose of carrageenan. Gel carriers, if used, may derive their gel consistency from a natural or synthetic binders, thickeners or gelling agents.

The orally compatible carrier 104 may further include one or more preservatives. Preservatives suitable for use in the present disclosure may include the natural preservative sodium gluconate. Sodium gluconate is used to prevent bacterial growth on the organic binders and humectants. It also works as a chelator (or sequestrant) which helps the composition to foam in hard water. Other preservative agents may include alcohols, benzoates, formaldehyde, and dichlorinated phenols.

The orally compatible carrier 104 may further include one or more foaming agents. The foaming agent in the present composition serves multiple functions. It may help to emulsify and evenly maintain the flavor oils throughout the product in the tube. When brushing, the foaming action produced may assist in the distribution of the product and then assists in the removal of debris from the mouth to ensure thorough rinsing. Foaming agents suitable for use in the present disclosure may include cocamidopropyl betain.

The orally compatible carrier 104 may include additional therapeutic agents such as, for example, zinc citrate used to reduce oral bacteria in the mouth. Plaque-producing bacteria may collect on teeth and gums resulting in gingivitis. Zinc inhibits the production of the bacteria that contribute to gingivitis.

The orally compatible carrier 104 may include one or more PH modifiers or buffers. PH modifiers or buffers suitable for use in the present disclosure may include sodium hydroxide. Dilute levels of sodium hydroxide may be used to adjust the pH level to improve the overall efficacy of the composition. When dissolved, the hydroxide portion of the sodium hydroxide reacts with acids to form water. The pH becomes more basic as some of the acid is eliminated.

The orally compatible carrier 104 may include one or more colorants. Colorants suitable for use in the present disclosure may include titanium dioxide. Titanium dioxide is a mineral-based white pigment used in very small concentrations in the composition to make the color of composition white, which makes it a more attractive product to many consumers. Other colorants may be used, for example, to differentiate the composition from other commercially available products.

Generally, no specific manufacturing guidelines or techniques are needed to prepare the composition. Suitable manufacturing techniques to produce the compositions are known. Notwithstanding, preparation of the compositions is not a matter of simple mixing or routine experimentation.

The manner of implementing the present disclosure will be illustrated further by the following examples. Although the following examples are intended to illustrate some features and advantages of the invention, they are not intended to limit the scope of the appended claims.

### Example 1—Oral Hygiene Composition

In a preferred embodiment of the oral hygiene composition 100, the orally compatible carrier 104 may be combined with an admixture 105 consisting essentially of a therapeutically effective amount of SnF₂ (stannous fluoride 106) and a therapeutically effective amount of tea tree oil 108. The range of such therapeutically effective amounts of SnF₂ (stannous fluoride 106) and tea tree oil 108 are shown in TABLE 2.

TABLE 2
Composition of Admixture 105

Percentage Amount

| Component | Minimum % | Maximum % |
|---|---|---|
| Stannous fluoride 106 | 0.01 | 99.9 |
| Tea Tree Oil 108 | 0.01 | 99.9 |

(Percentages by volume)

### Example 2—Oral Hygiene Composition

In another preferred embodiment of the oral hygiene composition 100, the orally compatible carrier 104 may be combined with an admixture 105 consisting essentially of specific therapeutically effective amounts of SnF₂ (stannous fluoride 106) and tea tree oil 108. Such therapeutically effective amounts of SnF₂ (stannous fluoride 106) and tea tree oil 108 are shown in TABLE 3.

TABLE 3
Composition of Admixture 105

Percentage Amount

| Component | Minimum % | Maximum % |
|---|---|---|
| Stannous fluoride 106 | 0.454 | 0.454 |
| Tea Tree Oil 108 | 1 | 5 |

(Percentages by volume)

The orally compatible carrier 104 of EXAMPLE 2 may further include one or more of the constituent components shown in TABLE 4.

TABLE 4
Composition of the orally compatible carrier 104
Component

Water
Sorbitol
Hydrated Silica
Sodium Lauryl Sulfate
Carrageenan
Sodium Gluconate
Flavorant
Xanthan Gum
Cocamidopropyl Betain
Zinc Citrate
Sodium Hydroxide
Sucralose (Sugar Substitute)
Titanium Dioxide

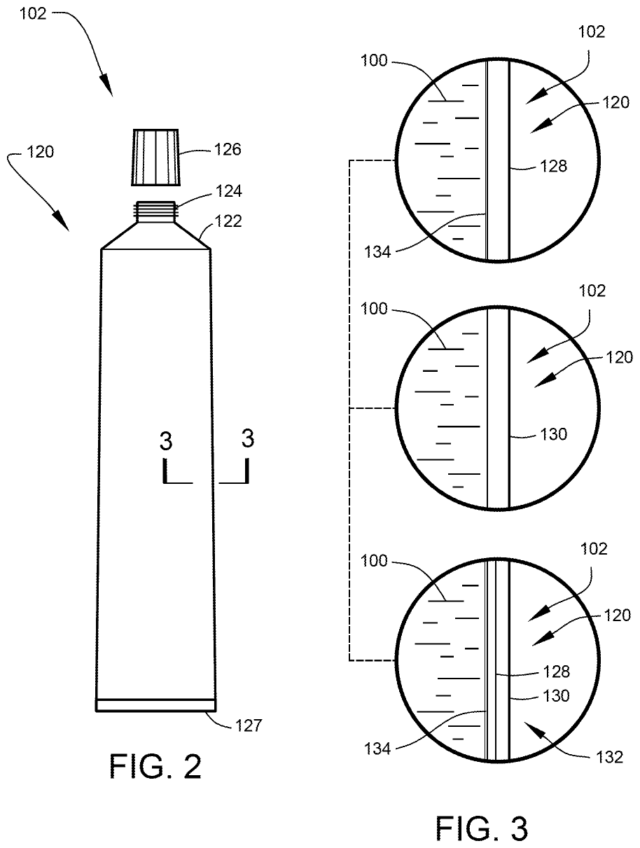

FIG. 2 shows a front view of product packaging 102 configured to package the oral hygiene composition 100 of FIG. 1, according to an embodiment of the present disclosure. FIG. 3 shows three sectional views, enlarged for clarity of description, further illustrating options for the product packaging 102 of FIG. 2, according to embodiments of the present disclosure. More specifically, FIG. 3 shows three different wall arrangements suitable for use in the construction of the product packaging 102.

As illustrated in FIG. 2 and FIG. 3, the oral hygiene composition 100 may be packaged in a squeezable container 120 comprising a collapsible tube constructed from a flexible material or layered materials. The squeezable container 120 may have a customary cylindrical shape with a shoulder 122, nozzle 124 and closure 126 at one end; the opposing end 127 may be open to allow filling with the oral hygiene composition 100 before the opposing end 127 is permanently sealed to provide a fully enclosed hygienic product dispenser.

Suitable closures 126 may include plastic caps attachable to the nozzle 124 by threaded engagement. Upon reading this specification, it should be appreciated that, under appropriate circumstances, considering such issues as user preferences, design preference, marketing preferences, cost, available materials, technological advances, etc., other closure arrangements such as, for example, flip-top caps, manual pumps, one-way valves, break-off tips for single-use or sampling tubes, tamper resistant features, etc., may be sufficient.

In specific reference to FIG. 3, the walls of the squeezable container 120 may be constructed of aluminum 128, plastic 130, or a composite assembly 132 combining aluminum 128, plastic 130, and the like, as shown. The inside of the aluminum tubes may be lined with an internal coating 134, as shown. The internal coating 134 may be a food-grade epoxy-phenolic or other suitable food-grade material to insure chemical compatibility with the composition and maximum shelf life.

Plastics 130 suitable for use in the present disclosure may include Linear Low Density Polyethylene (LLDPE), Low Density Polyethylene (LDPE), Medium Density Polyethylene (MDPE), Ethylene Vinyl Alcohol Copolymer (EVOH). The plastic tubes may be single or multilayered. The thickness and density of each layer may be adjusted to control the tube's flexibility and barrier properties.

Squeezable containers 120 utilizing composite assemblies 132 may employ an inner metallic layer (aluminum 128) and one or more outer layers comprising plastic 130. Aluminum 128 is used as a gas and light impermeable barrier layer within the composite assembly. The outer layer of plastic 130 provides a suitable surface for receiving printed graphics. The outer layer of the squeezable container 120 may be printed with any selected graphics using one or more well-known printing processes.

The squeezable container 120 may be produced on standard machines in conventional operations. Those with ordinary skill in the art will now appreciate that upon reading this specification and by their understanding the art of product packaging as described herein, methods of manufacturing such squeezable containers will be understood by those knowledgeable in such art. Upon reading this specification, it should be appreciated that, under appropriate circumstances, considering such issues as user preferences, design preference, structural requirements, marketing preferences, cost, available materials, technological advances, etc., other packaging arrangements such as, for example, use of other natural and biodegradable materials, the use of paper materials, glass, alternate composite materials, alternate package shapes, etc., may be sufficient.

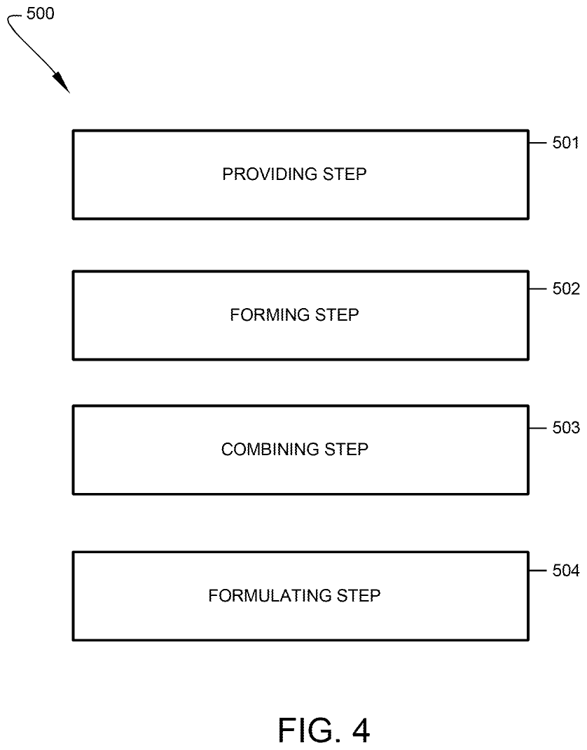

FIG. 4 is a flow diagram illustrating a method 500 of producing the oral hygiene composition 100, according to an embodiment of the present disclosure. As illustrated, the method 500 may include the steps of: step one 501, providing an aqueous orally compatible carrier 104; step two 502, forming an admixture 105 comprising essentially a therapeutically effective amount of SnF₂ (stannous fluoride 106) and a therapeutically effective amount of tea tree oil 108; and; step three 503, combining the admixture 105 and the aqueous orally compatible carrier 104 to form such oral hygiene composition 100. Even further, method 500 may include the additional step four 504, of formulating the aqueous orally compatible carrier 104 from at least one member selected from the group consisting essentially of: water; at least one sugar substitute; hydrated silica; sodium lauryl sulfate; carrageenan; sodium gluconate; at least one flavorant; xanthan gum; cocamidopropyl betain; zinc citrate; and titanium dioxide.

It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for oral hygiene composition 100 (e.g., different step orders within the above-mentioned list, elimination or addition of certain steps, including or excluding certain maintenance steps, etc.), are taught herein.

The embodiments of the disclosure described herein are exemplary and numerous modifications, variations and rearrangements can be readily envisioned to achieve substantially equivalent results, all of which are intended to be embraced within the spirit and scope of the disclosure. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. An oral hygiene composition for promoting oral health comprising:
an orally compatible carrier; and
an admixture consisting essentially of
a therapeutically effective amount of SnF₂ (stannous fluoride); and
a therapeutically effective amount of Melaleuca (tea tree oil).

2. The oral hygiene composition of claim 1, wherein the therapeutically effective amount of SnF₂ (stannous fluoride) comprises about 0.454 percent by volume.

3. The oral hygiene composition of claim 1, wherein the therapeutically effective amount of Melaleuca (tea tree oil) comprises between about 1 percent and about 5 percent by volume.

4. The oral hygiene composition of claim 1, wherein the orally compatible carrier comprises a semi-liquid aqueous paste.

5. The oral hygiene composition of claim 1, wherein the orally compatible carrier comprises
at least one sugar substitute selected from the group consisting essentially of sorbitol, saccharin, and sucralose.

6. The oral hygiene composition of claim 1, wherein the orally compatible carrier comprises at least one flavorant.

7. The oral hygiene composition of claim 1, wherein the orally compatible carrier comprises hydrated silica.

8. The oral hygiene composition of claim 1, wherein the orally compatible carrier comprises sodium lauryl sulfate.

9. The oral hygiene composition of claim 1, wherein the orally compatible carrier comprises carrageenan.

10. The oral hygiene composition of claim 1, wherein the orally compatible carrier comprises sodium gluconate.

11. The oral hygiene composition of claim 1, wherein the orally compatible carrier comprises xanthan gum.

12. The oral hygiene composition of claim 1, wherein the orally compatible carrier comprises cocamidopropyl betain.

13. The oral hygiene composition of claim 1, wherein the orally compatible carrier comprises zinc citrate.

14. The oral hygiene composition of claim 1, wherein the orally compatible carrier comprises sodium hydroxide

15. The oral hygiene composition of claim 1, wherein the orally compatible carrier comprises titanium dioxide

16. The oral hygiene composition of claim 1 further comprising
packaging configured to package the oral hygiene composition;
wherein the packaging comprises at least one of
a squeezable plastic tube,
a squeezable aluminum tube, and
a squeezable laminated tube comprising aluminum and plastic.

18. An oral hygiene composition for promoting oral health, the oral hygiene composition comprising:
a therapeutically effective amount of SnF₂ (stannous fluoride);
a therapeutically effective amount of Melaleuca (tea tree oil).
an orally compatible carrier configured to carry the SnF₂ and the tea tree oil;
water;
at least one sugar substitute;
hydrated silica;
sodium lauryl sulfate;
carrageenan;
sodium gluconate;
at least one flavorant;
xanthan gum;
cocamidopropyl betain;
zinc citrate; and
titanium dioxide; and
the oral hygiene composition forming an admixture consisting essentially of the SnF₂; the tea tree oil; the orally compatible carrier configured to carry the SnF₂ and the tea tree oil; water; the at least one sugar substitute; the hydrated silica; the sodium lauryl sulfate; the carrageenan; the sodium gluconate; the at least one flavorant; the xanthan gum; the cocamidopropyl betain; the zinc citrate; and the titanium dioxide.

19. A method of producing an oral hygiene composition, the method comprising the steps of:
providing an aqueous orally compatible carrier;
forming an admixture comprising a therapeutically effective amount of SnF₂ (stannous fluoride) and a therapeutically effective amount of tea tree oil; and
combining the admixture and the aqueous orally compatible carrier to form said oral hygiene composition.

20. The method of producing an oral hygiene composition of claim 19 further comprising the step of formulating the aqueous orally compatible carrier from at least one member selected from the group consisting essentially of:
water;
at least one sugar substitute;
hydrated silica;
sodium lauryl sulfate;
carrageenan;
sodium gluconate;
at least one flavorant;
xanthan gum;
cocamidopropyl betain;
zinc citrate; and
titanium dioxide.
